(19) United States
(12) Patent Application Publication
(10) Pub. No.: US 2024/0035063 A1
(43) Pub. Date: Feb. 1, 2024

(54) SYNTHETIC MIXED CULTURE RESEMBLING A SKIN MICROBIOME

(71) Applicant: Evonik Operations GmbH, Essen (DE)

(72) Inventors: Tobias BLATTERT, Essen (DE); Peter Lersch, Dinslaken (DE)

(73) Assignee: Evonik Operations GmbH, Essen (DE)

(21) Appl. No.: 18/255,344

(22) PCT Filed: Nov. 26, 2021
(86) PCT No.: PCT/EP2021/083112

§ 371 Date: May 31, 2023

(30) Foreign Application Priority Data
Dec. 1, 2020 (EP) 20210929.4

(51) Int. Cl. C12Q 1/02; C12N 1/20

(52) U.S. Cl. CPC C12Q 1/025; C12N 1/20; G01N 2500/10

(57) ABSTRACT

An in vitro method can be used for screening of the bioactivity of a compound by a synthetic mixed culture resembling a skin microbiome.

### FIELD OF THE INVENTION

The present invention relates to an in vitro method for screening of the bioactivity of a compound by means of a synthetic mixed culture resembling a skin microbiome.

### PRIOR ART

As the largest organ of the body, human skin is an extremely complex and dynamic substrate, whose function is to provide a physical barrier to injury and microbial insults. Today it is well established that skin is home to a large number and wide variety of microorganisms such as bacteria, fungi, and viruses. Each square centimeter of skin is inhabited by an estimated 1 million bacteria, with hundreds of distinct microbial species living together in mixed communities. The balanced microflora is in a dynamic equilibrium with the tissue (Holland, K. T., Bojar, R. A., Am. J. Clin. Dermatol., 2002, 3, 445-449) and can thus be regarded as an integral constituent of the skin. Through a number of mechanisms, the skin controls the fact that that microorganisms cannot spread indiscriminately and in particular a stop is put on pathogenic microorganisms. Those microbes and their genes make up the human skin microbiomes.

Studies have shown that gender, age, lifestyle, living area, genetic predisposition, diet and drug use (medication intake) are intricately related to the skin microbiome. Also, various stressors can promote metabolic changes within the cutaneous microenvironments.

Under specific conditions, the restriction of the colonization by pathogenic microorganisms such as pathogenic bacteria, yeasts and molds can be impaired and causes interferences in the cutaneous microflora. Colonization by such microorganisms can upset the balance of a healthy microbiome. When the balance in these communities is severely disrupted—a state termed as “dysbiosis”—a negative impact on the condition of the skin and human health of the host can be observed.

By comparing site-specific dermal microbiomes in healthy and diseased states, several studies have clearly demonstrated that disease is associated with perturbations of the composition of the microbiota and its metagenome.

In healthy skin a large variety of microorganisms can be found, whereas the diversity of the microbiome often decreases with abnormal skin conditions. This has been studied in patients with atopic dermatitis, where a significantly different and less diverse bacterial colonization pattern in the skin is observed—not only in affects areas with acute or chronic eczema, but in non-inflamed skin areas as well.

EP3049533 thus discloses the characterization of the bacterial signature associated with atopic dermatitis and the use thereof in in vitro methods for prognosis and/or diagnosis of atopic dermatitis, methods for monitoring response to a treatment, methods for monitoring the development of atopic dermatitis, as well as methods for selecting compounds useful in the prevention and/or treatment of atopic dermatitis.

Both frequent washing and the use of specific cosmetics have been previously implicated with modifying the skin microbiome.

Bouslimani et al. disclosed in BMC Biology 2019, 17:47 the impact of skin care products on skin chemistry and microbiome dynamics.

Wallen-Russell discloses in Cosmetics 2019, 6, 2 an analysis on the role of regularly used cosmetics in altering the skin microbiome.

Furthermore, a method has been disclosed in EP2776836 for identifying test agents that exhibit prebiotic activity on human skin commensal microorganisms and compositions that include such agents. The method includes providing a test culture of a test agent, a human skin commensal microorganism and a minimal carbon medium.

It is a disadvantage of all prior art, that the disclosed methods are either carried out in a skin substrate and take long period of times to come to results or that the methods do not even come close to the complexity of a real skin microbiome due to the very limited amount of different microbes.

It is an object of the invention to provide a method for rapid and reproducible testing of compounds also resembling the complexity of natural occurring skin microbiomes.

### DESCRIPTION OF THE INVENTION

It was found that, surprisingly, that the method of claim 1 is able to provide a solution for the object of the instant invention and overcomes at least one of the disadvantages of the prior art.

The present invention therefore provides an in vitro method for screening of the bioactivity of a compound by means of a synthetic mixed culture resembling a skin microbiome as described in claim 1.

The invention further provides a synthetic mixed culture resembling a skin microbiome as described in claim 11.

One advantage of the present invention is that the method of the instant invention delivers reliable results within hours.

Another advantage of the present invention is that it is able to mimic the microbiome of natural, healthy human skin and its changes due to externally applied compounds.

A further advantage of the present invention is that the microbiome of different body areas can be mimicked efficiently.

Another advantage of the present invention is, that resources are saved as the method of the invention can be carried out with minimal sample volumes.

A further advantage of the present invention is, that no skin or skin like substrates are necessary.

Another advantage of the present invention is the representation of well-defined and most controllable environments for mechanistic and molecular profiling.

A further advantage of the present invention is that fluctuations and changes in the measured microbial communities resulting from gross differences in population structure, geography, or environmental conditions can be avoided.

Another advantage of the present invention is that issues with the collection and processing of low-biomass skin microbiome samples can be extremely simplified.

A further advantage of the present invention is that the risk of contaminations can be greatly reduced.

An aspect of the instant invention thus is an in vitro method for screening of the bioactivity of a compound, said method comprising the steps of
a) providing a synthetic mixed culture of at least 8, preferably at least 10, more preferably at least 12, different bacterial strains resembling a skin microbiome,
b) cultivating said synthetic mixed culture in a medium comprising the compound over a period of time,
c) measuring the diversity level and/or the diversity profile of said synthetic mixed culture after step b) and comparing the diversity level and/or the diversity profile of said synthetic mixed culture to the diversity level and/or the diversity profile of a control microbiome, and
d) deducing the bioactivity of the compound by the deviations between said synthetic mixed culture and said control microbiome obtained in step c).

The term “bioactivity” in context with the instant invention comprises the effect of the compound on the organisms contained in the synthetic mixed culture during cultivation in terms of how the relative abundance of the microorganisms alter.

By analysing this kind of bioactivity of a compound, one can deduct the effect of the compound on skin in vivo, as the role and effect of different microorganisms within a skin microbiome is known.

Thus, the term “bioactivity of a compound” in context with the instant invention comprises the cosmetic effect on skin of the compound.

Examples for cosmetic effects are, for example, improvement of skin appearance, de-fattening of skin, fattening of skin, firming of skin, compensation of irregular pigmentation or wrinkling of the skin, smoothing of skin, skin protection against UV irradiation and photoaging, revitalizing the skin, enhancing skin moisturization and the prevention of dehydration, addressing skin sensitivity and mitigating environmental influences that can lead to negative skin conditions such as skin aging.

Unless stated otherwise, all percentages (%) given are percentages by mass.

Preferably the compound, whose bioactivity is screened for is a cosmetic ingredient.

Any kind of cosmetic ingredient can be screened in the method according to the instant invention. Examples are plant extracts, botanical ingredients and phytochemicals, emollients, emulsifiers, thickeners, UV light protection filters, antioxidants, hydrotropes, polyols, solids, fillers, film formers, pearlescence additives, deodorant and antiperspirant active ingredients, insect repellents, self-tanning agents, preservatives, conditioning agents, rheology modifiers, colorants, dyes, odor absorbers, superfatting agents, solvents and surfactants.

Preferably the compound, whose bioactivity is screened for is selected from the group of preservatives, plant extracts and botanical ingredients, UV light protection filters, emulsifiers and surfactants.

The term “synthetic mixed culture” in context with the instant invention comprises a mixture of microorganisms that have been isolated before from natural sources, so called “isolated microorganisms”. This term implies, that different and separately isolated microorganisms were combined (“mixed”) to result in the synthetic mixed culture.

The term “resembling a skin microbiome” in context with the instant invention is to be understood, that the synthetic mixed culture's properties are very similar to a natural occurring microbiome present on skin, although its complexity in terms of number of different microorganisms is reduced compared to the natural occurring microbiome present on the respective skin.

Preferably the skin microbiome resembled in step a) of the method according to the instant invention is the skin microbiome of a healthy human individual.

In this instance screening of the bioactivity of a compound is preferably conducted to assess, whether negative or positive impacts on a healthy human individual's skin are found.

Alternatively preferred the skin microbiome resembled in step a) of the method according to the instant invention is the skin microbiome of a human individual suffering from a skin disorder associated with a specific skin microbiome. Such disorders include, but are not limited to, seborrheic dermatitis, atopic dermatitis, psoriasis, acne vulgaris and hidradenitis suppurativa. In this context, screening of the bioactivity of a compound is preferably conducted to assess, whether positive impacts on the human individual's skin microbiome, who is suffering from a skin disorder skin, is found, thus improving the condition of the associated disorder.

Any bacterial strain known to be part of a skin microbiome, preferably of a human skin microbiome, can be used as a bacterial strain resembling the skin microbiome according to the instant invention. Exemplary skin commensal bacteria include, but are not limited to, Alphaproteobacteria, Betaproteobacteria, Gammaproteobacteria, Propionibacteria, Corynebacteria, Actinobacteria, Clostridiales, Lactobacillales, Staphylococcus, Bacillus, Micrococcus, Streptococcus, Bacteroidales, Flavobacteriales, Enterococcus and Pseudomonas.

The bacterial strains resembling the skin microbiome are preferably selected from the group comprising, preferably consisting of, Cutibacterium acnes, Corynebacterium afermentans, Corynebacterium amycolatum, Corynebacterium aurimucosum, Corynebacterium fastidiosum, Corynebacterium kroppenstedtii, Corynebacterium resistens, Corynebacterium simulans, Corynebacterium striatum, Corynebacterium tuberculostearicum, Corynebacterium xerosis, Enhydrobacter aerosaccus, Micrococcus luteus, Propionibacterium acnes, Staphylococcus aureus, Staphylococcus capitis, Staphylococcus epidermidis, Staphylococcus haemolyticus, Staphylococcus hominis, Staphylococcus warneri. Streptococcus mitis, Streptococcus oralis, Streptococcus pseudopneumoniae, Streptococcus sanguinis, and Veillonella parvula.

Skin can be physiologically grouped into different sites:

For example, hypothenar palm, and volar forearm are considered as dry sites, nare, antecubital fossa, inguinal crease, interdigital web, popliteal fossa are considered moist sites, alar crease, cheek, glabella, external auditory canal, manubrium, retroauricular crease, occiput, and back are considered as sebaceous sites, and toe web space, toenail and plantar heel are considered as foot sites.

When resembling the skin microbiome of different skin sites in step a) of the method of the instant invention it is preferred, that in case a dry site is resembled, the bacterial strains resembling the skin microbiome are preferably selected from the group comprising, preferably consisting of,
A) Corynebacterium tuberculostearicum, Micrococcus luteus, Propionibacterium acnes, Staphylococcus capitis, Staphylococcus epidermidis, Streptococcus mitis, Streptococcus oralis, Streptococcus pseudopneumoniae, Streptococcus sanguinis, and Veillonella parvula, in case a moist site is resembled, the bacterial strains resembling the skin microbiome are preferably selected from the group comprising, preferably consisting of,
B) Corynebacterium afermentans, Corynebacterium fastidiosum, Corynebacterium simulans, Corynebacterium tuberculostearicum, Enhydrobacter aerosaccus, Micrococcus luteus, Propionibacterium acnes, Staphylococcus capitis, Staphylococcus epidermidis and Staphylococcus hominis, in case a sebaceous site is resembled, the bacterial strains resembling the skin microbiome are preferably selected from the group comprising, preferably consisting of,
C) Corynebacterium amycolatum, Corynebacterium aurimucosum, Corynebacterium kroppenstedtii, Corynebacterium simulans, Corynebacterium tuberculostearicum, Propionibacterium acnes, Staphylococcus capitis, Staphylococcus epidermidis, Staphylococcus hominis and Streptococcus mitis, in case a foot site is resembled, the bacterial strains resembling the skin microbiome are preferably selected from the group comprising, preferably consisting of,
D) Corynebacterium afermentans, Corynebacterium resistens, Corynebacterium simulans, Corynebacterium tuberculostearicum, Micrococcus luteus, Staphylococcus capitis, Staphylococcus epidermidis, Staphylococcus haemolyticus, Staphylococcus hominis and Staphylococcus warneri.

The bacterial strains resembling the skin microbiome are preferably selected from the group comprising, preferably consisting of, Cutibacterium acnes, Corynebacterium afermentans, Corynebacterium tuberculostearicum, Corynebacterium xerosi, Micrococcus luteus, Staphylococcus aureus, Staphylococcus capitis,

Staphylococcus epidermidis Staphylococcus hominis, Staphylococcus warneri and Streptococcus mitis.

The synthetic mixed culture of the instant invention is not limited to bacteria species only, as the microbiome of the human skin also contains eukaryotic and viral species.

Thus, it is preferred according to the instant invention, that the synthetic mixed culture in step a) of the instant invention comprises eukaryotic and/or viral species, preferably eukaryotic species, with Malassezia, Aspergillus, Debaroyomyces, and Cryptococcus being preferred, and Malassezia, with a preference for M. restricta and M. globosa, being most preferred.

It is an advantage of the method of the instant invention, and thus preferred, that cultivation in step b) can be conducted with the bacterial strains resembling the skin microbiome being at least in part in suspension. It has been surprisingly found, that the growth of bacterial strains resembling the skin microbiome is faster than compared to their growth on substrates while still undergoing the same changes in diversity level and/or diversity profile.

The cultivation in step b) according to the instant invention can be carried out under aeration, under reduced aeration or without any aeration.

Depending on the grade of aeration, which can be influenced for example by provision of oxygen gas and or the degree of agitation of the medium, the cultivation is considered to be aerobic or anaerobic.

The term “anaerobic cultivation” in context with the instant invention means a cultivation in the presence of the gas in contact with cultivation medium having a concentration of less than 5 vol. % of oxygen.

The term “aerobic cultivation” in context with the instant invention means a in the presence of the gas in contact with cultivation medium having a concentration of 5 or more vol. % of oxygen.

A preferred method according to the instant invention is characterized in, that cultivation in step b) is conducted anaerobically, more preferred with the provision of nitrogen to the gas in contact with the medium.

A alternatively preferred method according to the instant invention is characterized in, that cultivation in step b) is conducted aerobically at low agitation rates, preferably at agitation rates of less than 800 rpm, more preferably at agitation rates of from 50 rpm to 700 rpm, most preferably at agitation rates of from 300 rpm to 600 rpm.

A preferred method according to the instant invention is characterized in, that cultivation in step b) is conducted in laboratory dishes, which preferably are selected from multi well plates, preferably from 6-, 24-, 48-, 96- and 384 well plates.

A preferred method according to the instant invention is characterized in, that cultivation in step b) is conducted over a period of time of from 2 hours to 48 hours, preferably from 4 hours to 36 hours, more preferably from 6 hours to 24 hours, even more preferably from 8 hours to 20 hours.

Different ways of measuring the diversity level and/or the diversity profile of a microbiome are well known to a person skilled in the art. They range from Sanger-sequencing, 16S rRNA analysis, over transcribed spacer 1 (ITS1) region analysis to whole genome sequencing, the latter capturing the entire complement of genetic material in a microbiome without a targeted amplification step. 16S rRNA analysis of microbiomes to assess the diversity of the sample is disclosed, for example in WO2020150723. A transcribed spacer 1 region analysis of microbiomes to assess the diversity of the sample is disclosed, for example in WO2015170979. Oh et al. in Cell 165,854-866 (2016) disclose suitable methods to assess the diversity of a microbiome by shotgun metagenomic sequencing.

A preferred method according to the instant invention is characterized in, that the diversity level and/or the diversity profile of the microbiome is measured by 16S rRNA analysis. This is especially true, when the synthetic mixed culture only contains bacterial strains as microorganisms resembling the skin microbiome.

In case the synthetic mixed culture of the instant invention comprises eukaryotic and/or viral species, the diversity level and/or the diversity profile of the microbiome is preferably measured by shotgun metagenomic sequencing

Step c) if the method according the instant invention comprises comparing the diversity level and/or the diversity profile of said synthetic mixed culture to the diversity level and/or the diversity profile of a control microbiome.

This includes preferably to determine the relative abundance of the microorganisms comprised in the synthetic mixed culture and the control microbiome and to determine the deviations in relative abundance between the two cultures.

A preferred method according to the instant invention is characterized in, that the control microbiome in step c) is selected from a synthetic mixed reference culture cultivated identically to said synthetic mixed culture but without the compound and said synthetic mixed culture at the beginning of step b), preferably it is a synthetic mixed reference culture cultivated identically to said synthetic mixed culture but without the compound.

Step d) if the method according the instant invention comprises deducing the bioactivity of the compound by the deviations between said synthetic mixed culture and said control microbiome obtained in step c).

A person skilled in the art will readily understand, what bioactivity the screened compound has, when the alteration of relative abundance of species in the synthetic mixed culture has been determined:

For example, if the microbial composition of the synthetic mixed culture differs upon exposure of a botanical or microbial extract in a way that the microbial species associated with any given skin disease are suppressed in their growth rate, whereas the commensal microbial species show a higher growth, the method can be used as an early diagnostic marker for compounds that are likely to have beneficial effect in the treatment of said skin disease.

A review article describing the associations on human skin between altered microbial communities and disease was published by L. Weyrich et al. in 2015 in Australasian Journal of Dermatology 56(4).

Evonik is commercially offering the product Skinolance®, which is a cell-free extract based on a natural probiotic Lactobacillus designed to keep and restore the natural balance of the skin microbiota ® fosters the growth of the beneficial bacterial Staphylococcus epidermidis while preventing the growth of Staphylococcus aureus, which is associated with dermatitis and dry skin. In the method of the instant invention this change in relative abundance of these two bacteria strains can be shown within hours.

Another aspect of the instant invention is a synthetic mixed culture of at least 8, preferably at least 10, more preferably at least 12, isolated, different bacterial strains resembling a skin microbiome.

Preferred embodiments of the synthetic mixed culture of the instant invention are describes above as preferred synthetic mixed culture provided in step a) of the method of the instant invention.

### BRIEF DESCRIPTION OF THE DRAWINGS

FIG. 1: Model microbiome composition at T=28 h in a non-treated, aerobic cultivation upon inoculation with strain mix according to ratio 1

FIG. 2: Model microbiome composition at T=22 h in a non-treated, anaerobic cultivation upon inoculation with strain mix according to ratio 1

FIG. 3: Model microbiome compositions at T=28 h in a non-treated, aerobic cultivation upon inoculation with strain mix according to ratio 1 compared with final compositions of cultures, which were treated with compound W, X, Y or Z.

FIG. 4: Model microbiome compositions at T=22 h in a non-treated, anaerobic cultivation upon inoculation with strain mix according to ratio 1 compared with final compositions of cultures, which were treated with compound W, X, Y or Z.

FIG. 5: Mean microbiome of five healthy human individuals prior treatment with Skinolance®.

FIG. 6: Mean microbiome of five healthy human individuals after treatment with Skinolance®.

The examples adduced hereinafter describe the present invention by way of example, without any intention that the invention, the scope of application of which is apparent from the entirety of the description and the claims, be restricted to the embodiments specified in the examples.

### EXAMPLES

### Example 1: Aerobic Cultivation of a Core Skin Microbiome Model

Individual bacterial strains are inoculated from cryocultures to initiate preculture growth. BHI Media (110493; Merck KGaA, 64293 Darmstadt) or DSMZ media 92 or DSMZ media 535 media according to the recommendations of the German Collection of Microorganisms and Cell Cultures GmbH are used for preculture cultivation.

Strains and growth conditions are summarized in table 1.

To avoid starvation, a single SMFB01001 glucose FeedBead (Kuhner Shaker GmbH, Kaiserstraße 100, 52134 Herzogenrath) is added to 10 ml of preculture medium if indicated.

TABLE 1
Preculture conditions of strains

| | Preculture conditions | | |
|---|---|---|---|
| Strain | Media | O₂ | Duration |
| Cutibacterium acnes | BHI (anaerobic aq) | Anaerobic, standing culture | 5 d |
| Streptococcus mitis | BHI | Aerobic, standing culture | 24 h |
| Corynebacterium | DSMZ 92 + 1 FeedBead | Aerobic, 200 rpm, Multitron | 48 h |
| afermentans | | HT shaker, Infors | |
| Staphylococcus capitis | BHI + 1 FeedBead | | 24 h |
| Staphylococcus epidermidis | | | |
| Staphylococcus hominis | | | |
| Staphylococcus warneri | | | |
| Staphylococcus aureus | | | |
| Micrococcus luteus | | | |
| Corynebacterium xerosis | | | |
| Corynebacterium | DSMZ 535 + 0.2% Tween | | |
| tuberculostearicum | 80 + 1 Kuhner bead | | |

At the end of cultivation strains are diluted to result in an optical density of OD₆₀₀=1 using BHI media. These single strain cultures are mixed in defined strain ratios depending on their growth properties. Example ratios, which lead to highly diverse cultures are summarized in table 2.

TABLE 2
Share of single strain cultures in different starting communities [Ratio 1-12]

| % | Ratio 1 | Ratio 2 | Ratio 3 | Ratio 4 | Ratio 5 | Ratio 6 |
|---|---|---|---|---|---|---|
| Cutibacterium acnes | 36.1 | 36.3 | 35.3 | 35.0 | 33.4 | 46.8 |
| Corynebacterium afermentans | 15.1 | 15.2 | 14.7 | 14.6 | 14.0 | 17.5 |
| Streptococcus mitis | 26.6 | 26.8 | 26.0 | 25.8 | 24.6 | 16.7 |
| Micrococcus luteus | 9.2 | 9.2 | 8.9 | 8.9 | 12.9 | 8.8 |
| Staphylococcus hominis | 3.2 | 3.2 | 3.1 | 3.1 | 2.9 | 2.0 |
| Staphylococcus warneri | 1.0 | 1.0 | 1.0 | 1.0 | 0.9 | 0.6 |
| Staphylococcus capitis | 2.9 | 2.9 | 2.8 | 3.6 | 3.4 | 2.3 |
| Staphylococcus epidermidis | 4.4 | 4.5 | 7.3 | 7.2 | 6.9 | 4.7 |
| Staphylococcus aureus | 1.5 | 0.9 | 0.9 | 0.9 | 0.9 | 0.6 |
| Corynebacterium tuberculostearicum | 0 | 0 | 0 | 0 | 0 | 0 |
| Corynebacterium xerosis | 0 | 0 | 0 | 0 | 0 | 0 |
| % | Ratio 7 | Ratio 8 | Ratio 9 | Ratio 10 | Ratio 11 | Ratio 12 |
| Cutibacterium acnes | 35.0 | 31.5 | 36.8 | 33.2 | 36.1 | 36.5 |
| Corynebacterium afermentans | 13.1 | 13.1 | 13.8 | 12.4 | 15.1 | 15.2 |
| Streptococcus mitis | 23.2 | 23.2 | 9.2 | 8.3 | 26.6 | 26.9 |
| Micrococcus luteus | 8.0 | 8.0 | 9.2 | 8.3 | 9.2 | 9.3 |
| Staphylococcus hominis | 2.8 | 2.8 | 9.2 | 11.6 | 3.2 | 3.2 |
| Staphylococcus warneri | 0.9 | 0.9 | 1.8 | 1.7 | 1.0 | 0.0 |
| Staphylococcus capitis | 3.2 | 3.2 | 9.2 | 11.6 | 2.9 | 2.9 |
| Staphylococcus epidermidis | 6.5 | 6.5 | 9.2 | 11.6 | 4.4 | 4.5 |
| Staphylococcus aureus | 0.8 | 0.8 | 1.5 | 1.3 | 1.5 | 1.5 |
| Corynebacterium tuberculostearicum | 10.0 | 0.0 | 0 | 0 | 0 | 0 |
| Corynebacterium xerosis | 0.0 | 10.0 | 0 | 0 | 0 | 0 |

Cultivation is carried out using a BioLector [m2p-labs; Arnold-Sommerfeld-Ring 2, 52499 Baesweiler] small scale test system with MTP-48-BOH 1 plates.

100 μl of strain mix, according to the ratios in table 2 are added to 1 ml BHI media in resulting in a starting OD₆₀₀ of 0.091. Cultivation is done at 37° C., a shaking frequency of 600 rpm and relative humidity of 85% for 28 h until the process is stopped.

At the end of cultivation OD₆₀₀ is determined, and the remaining culture is centrifuged at 4° C., 5000 rpm and 5 min in an Eppendorf table centrifuge. The supernatant is discarded, and the pellet is stored at −20° C.

Genomic DNA is extracted from these samples using the DNeasy PowerSoil Pro Kit [Qiagen GmbH, QIAGEN Strasse 1, 40724 Hilden]. DNA samples are analyzed by LGC Genomics GmbH [Ostendstraße 25, 12459 Berlin] using primer pair 341 F, Seq. ID NO 1, and 785R, Seq. ID NO 2, or library preparation and !lumina MiSeq for sequencing.

Results are processed by LGC Genomics GmbH and mapped to an individual reference database containing the sequences of the strains which are present in the individual model.

### Example 2: Anaerobic Cultivation of a Core Skin Microbiome Model

Preculture cultivation and ratio mixing is carried out as in example 1. Anaerobic cultivation is done using a BioLector [m2p-labs; Arnold-Sommerfeld-Ring 2, 52499 Baesweiler] small scale test system with MTP-48-BOH 1 plates and the anaerobic module including an external mass flow controller for nitrogen (N₂).

100 μl of strain mix, according to the ratios in table 2 are added to 1 ml BHI media resulting in a starting OD₆₀₀ of 0.091. Cultivation is done at 37° C., a shaking frequency of 600 rpm and under nitrogen flushing for 22 h until the process is stopped.

### Example 3: Characterization of the Influence of Test Compounds on the Core Microbiome Model

Preculture cultivation, ratio mixing and cultivation is carried out as in example 1 or 2. Test compounds are added to the cultures at T=0, 4, 8, 12, 16, or 24 h. At the end of the cultivation samples are taken and processed as in example 1 or 2 and results are compared to non-treated cultures to evaluate the effect of the test compounds on the core microbiome model.

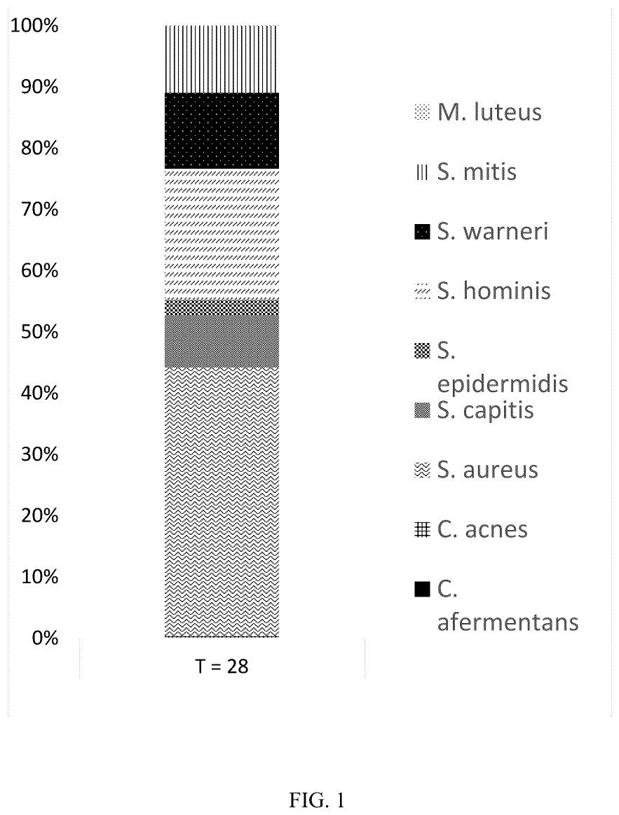

Cultivation of Ratio 1 under aerobic condition results in an optical density of OD₆₀₀=17.2 and a final microbiome model composition as shown in FIG. 1.

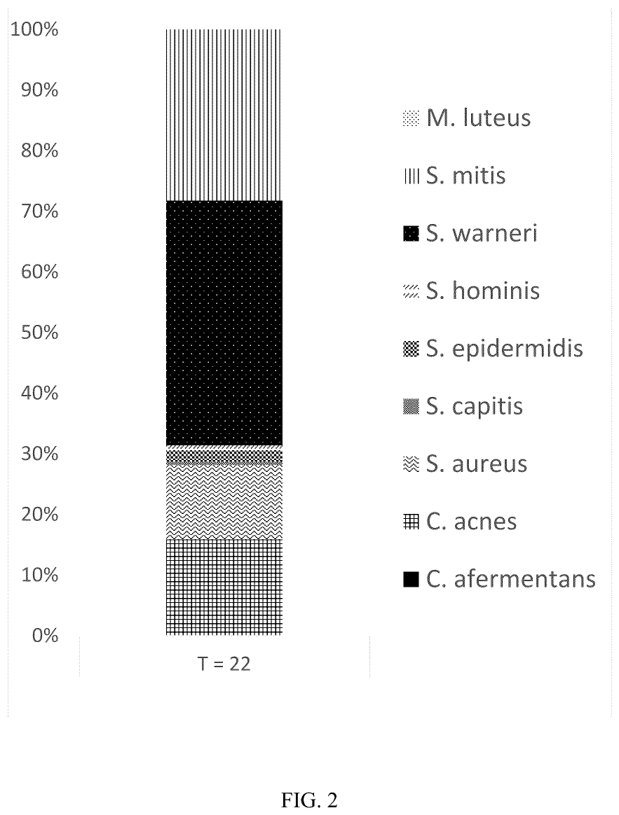

Cultivation of Ratio 1 under anaerobic condition results in an optical density of OD₆₀₀=1.81 and a final microbiome model composition as shown in FIG. 2.

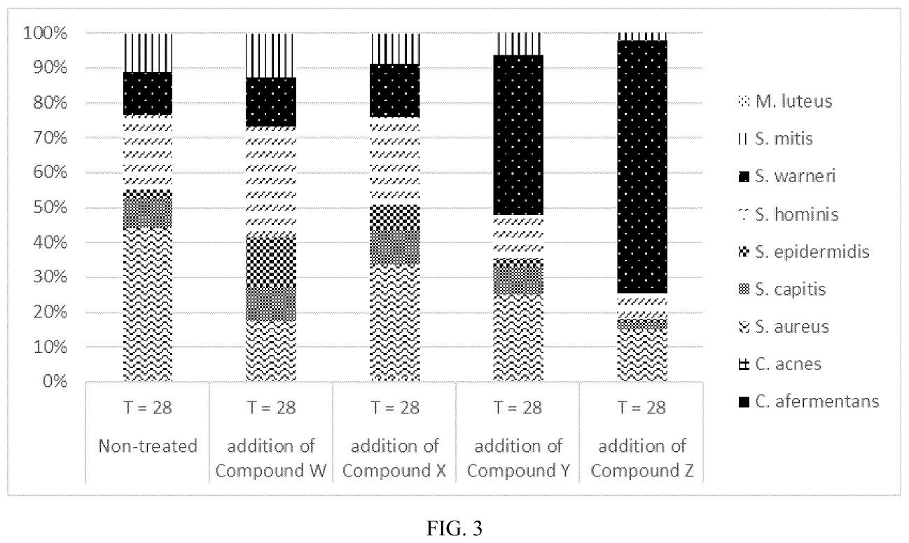

Upon treatment of an aerobically cultivated culture, which is inoculated with ratio 1 and treated with compounds Skinolance®, Evonik (W), TEGO® Cosmo C 100 (Creatine) from Evonik (X), cell-free lysate of Pseudomonas Putida (Y) or TEXAPON® NSO (sodium lauryl ether sulfate) from BASF (Z) at T=0, the final microbiome compositions vary significantly as shown in FIG. 3.

Skinolance® causes a remarked reduction of S. aureus in our model, while increasing the abundance of S. epidermidis and keeping the microbial diversity at an unchanged, high level. Due to its pronounced reduction of S. aureus, Skinolance® can be considered a microbiome-positive compound with apparent benefits for the treatment of atopic and/or dry skin conditions, specifically. Only a minor shift compared to the baseline composition is obtained with Creatine, a revitalizer of the cellular energy metabolism, under aerobic conditions: Only the relative abundance of both S. epidermidis and S. hominis—which have been associated with skin health—are slightly increased and S. aureus was correspondingly slightly decreased. Therefore, Creatine can be considered as a microbiome-neutral compound.

P. putida lysate has a bigger effect on the baseline shift, with a remarked reduction of all species besides no effect on S. capitis and a corresponding increased abundance of S. warneri. Due to its pronounced reduction of S. aureus, this lysate can be considered a microbiome-positive compound.

Treatment with the harsh surfactant sodium lauryl ether sulfate is causing a drastic microbial community shifts with a significantly increased abundance of S. warneri at the expense of all other Staphylococcus species, i.e. a remarked loss of diversity is observed. Since overgrowth of one species is often associated with pathogenic skin, the use of the pure compound on the skin might cause negative effects on the microbiome.

When comparing the growth pattern of the same starting culture under aerobic versus anaerobic conditions, surprisingly it is found that under aerobic conditions a mixed, Staphylococcus-dominant culture is formed, which can mimic both sebaceous sites and also represents the moist sites of human skin. These staphylococci play also an important role in skin protection by their ability to reduce the pathogen load on the skin surface and maintain the community structure on the skin surface effectively.

A different but also reproducible synthetic model community is obtained under anaerobic conditions. We suspect that the reason for this effect is due to the discriminatory effect which oxygen has on each of the members, which will affect the growth pattern of the microbial model community. Under anaerobic culture conditions, a better inclusion of Cutibacterium acnes into the synthetic mixed cultures is possible and this species can be grown in co-cultures in higher abundance together with Staphylococcus species including S. epidermidis and S. aurueus). C. acnes is well known as a contributor to the development of the skin disease, acne, although the mechanistic details of how C. acnes promotes acne are not well understood and C. acnes may not be involved in all cases of acne (Shaheen & Gonzalez, 2013). Since C. acnes preferentially inhabits sebum-rich skin regions and can consume skin oil (sebum) and produce byproducts such as short-chain fatty acids and propionic acid, which are known to help maintain a healthy skin barrier, this synthetic model community may be particularly useful for screening and predicting the in-vivo effects of potential ingredients that are beneficial for face applications targeted against e.g. oily and/or acne prone skin.

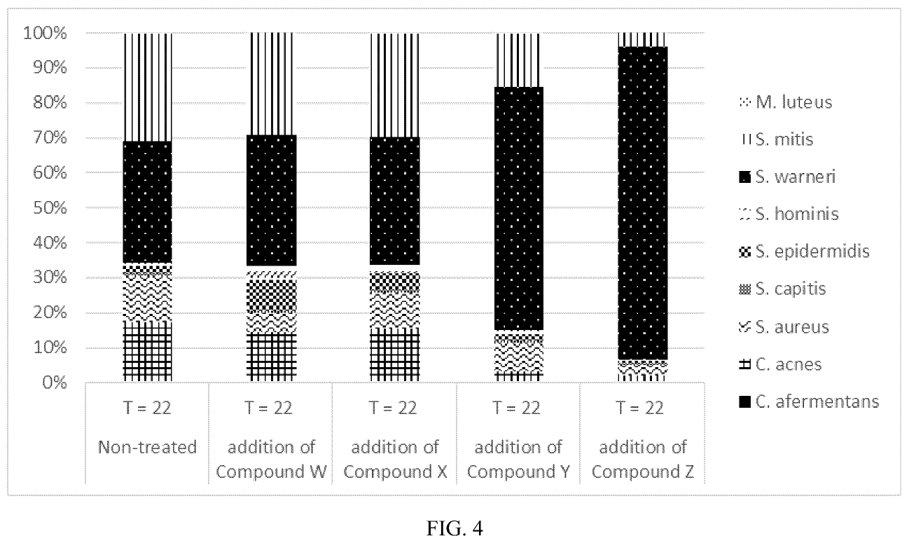

Upon treatment of an anaerobically cultivated culture, which is inoculated with ratio 1 and treated with compounds X, Y or Z at T=0, the final microbiome compositions vary significantly as shown in FIG. 4 due to the growth conditions for C. acnes. However, the general trends for the overall predictability of the synthetic community remain the same also under anaerobic conditions, as judged by the results obtained for the compounds Skinolance® (W), Creatine (X) and Sodium Lauryl ether sulfate (Z).

The most different result is obtained for the cell-free lysate of Pseudomonas putida (Y), which due to its versatile efficacy of lowering the abundance of C. acnes in the mixed synthetic culture while only slightly reducing the microbial diversity may be particularly useful for personal care products designed to inhibit the growth and proliferation of acne bacteria and cleansing products for oily skin areas.

### Example 4: Characterization of the Influence of Test Compounds on the Microbiome on Skin In Vivo

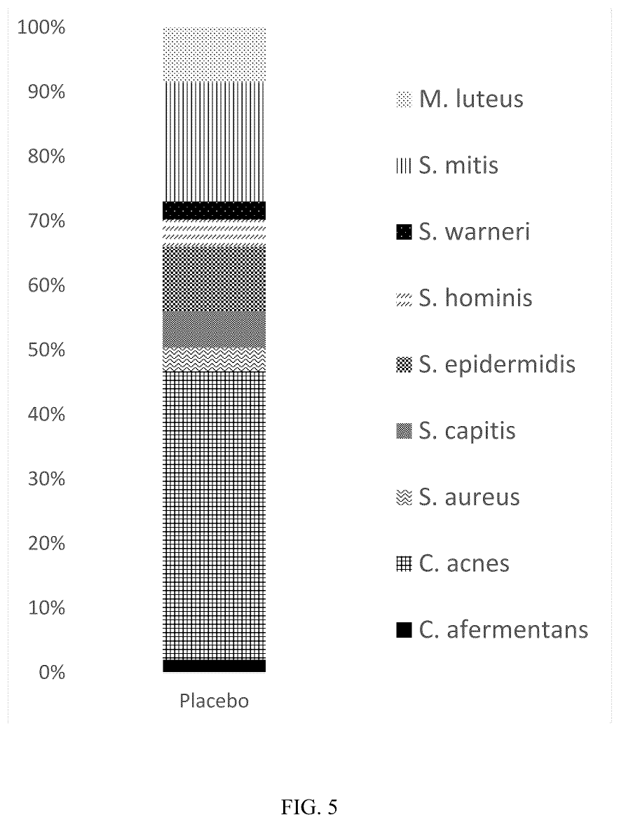

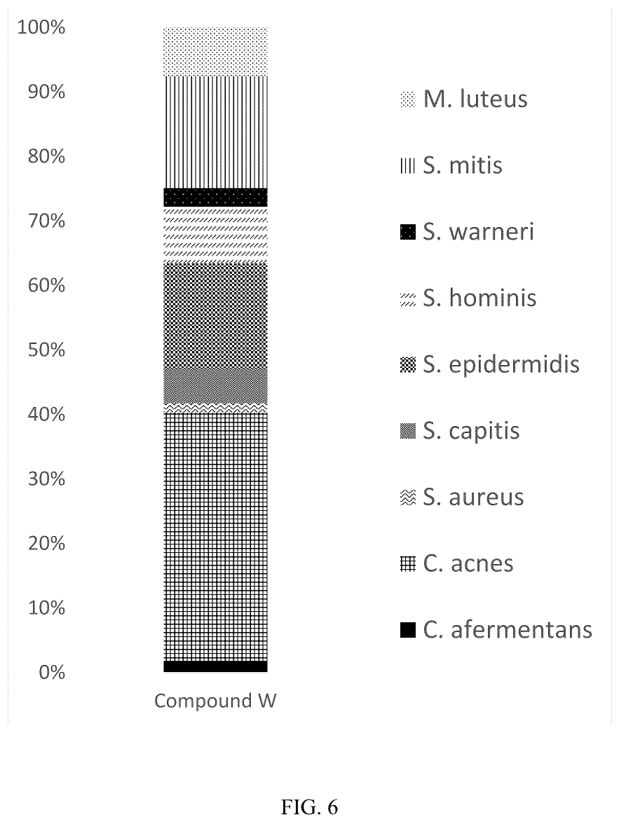

The microbiome of five human individuals from the lower leg region are analyzed for their composition and fifty-nine bacterial morphotypes can be isolated. From the species identified, the same eleven species are selected from the previously obtained in vitro synthetic model communities (examples 1 to 3) and their relative abundance is calculated (FIG. 5). After treatment with a cosmetic O/W cream containing 2% Skinolance® (compound W) for 28 days with a daily dose on the lower leg against placebo, the individuals’ skin microbiomes are analyzed again. FIGS. 5 and 6 show mean averages of the test persons' microbiome compositions.

As shown in FIG. 5 and FIG. 6, the readout shows a similar increased abundance of S. epidermidis and S. hominis species together with a reduced abundance of S. aureus. Thereby, changes in the microbiome composition before and after treatment of the skin with Skinolance® can be clearly correlated to the changes also seen in example 3, demonstrating the practicability of the synthetic model communities.

1. An in vitro method for screening of the bioactivity of a compound, said method comprising:
a) providing a synthetic mixed culture of at least 8 different bacterial strains resembling a skin microbiome,
b) cultivating said synthetic mixed culture in a medium comprising the compound over a period of time,
c) measuring a diversity level and/or a diversity profile of said synthetic mixed culture after b) and comparing the diversity level and/or the diversity profile of said synthetic mixed culture to a diversity level and/or a diversity profile of a control microbiome, and
d) deducing the bioactivity of the compound by deviations between said synthetic mixed culture and said control microbiome obtained in c).

2. The method according to claim 1, wherein the bacterial strains are selected from the group consisting of Cutibacterium acnes, Corynebacterium afermentans, Corynebacterium amycolatum, Corynebacterium aurimucosum, Corynebacterium fastidiosum, Corynebacterium kroppenstedtii, Corynebacterium resistens, Corynebacterium simulans, Corynebacterium striatum, Corynebacterium tuberculostearicum, Corynebacterium xerosi, Enhydrobacter aerosaccus, Micrococcus luteus, Propionibacterium acnes, Staphylococcus aureus, Staphylococcus capitis, Staphylococcus epidermidis, Staphylococcus haemolyticus, Staphylococcus hominis, Staphylococcus warneri. Streptococcus mitis, Streptococcus oralis, Streptococcus pseudopneumoniae, Streptococcus sanguinis, and Veillonella parvula.

3. The method according to claim 1, wherein the bacterial strains are selected from the group consisting of
A) Corynebacterium tuberculostearicum, Micrococcus luteus, Propionibacterium acnes, Staphylococcus capitis, Staphylococcus epidermidis, Streptococcus mitis, Streptococcus oralis, Streptococcus pseudopneumoniae, Streptococcus sanguinis, and Veillonella parvula,
B) Corynebacterium afermentans, Corynebacterium fastidiosum, Corynebacterium simulans, Corynebacterium tuberculostearicum, Enhydrobacter aerosaccus, Micrococcus luteus, Propionibacterium acnes, Staphylococcus capitis, Staphylococcus epidermidis and Staphylococcus hominis,
C) Corynebacterium amycolatum, Corynebacterium aurimucosum, Corynebacterium kroppenstedtii, Corynebacterium simulans, Corynebacterium tuberculostearicum, Propionibacterium acnes, Staphylococcus capitis, Staphylococcus epidermidis, Staphylococcus hominis, and Streptococcus mitis,
D) Corynebacterium afermentans, Corynebacterium resistens, Corynebacterium simulans, Corynebacterium tuberculostearicum, Micrococcus luteus, Staphylococcus capitis, Staphylococcus epidermidis, Staphylococcus haemolyticus, Staphylococcus hominis, and Staphylococcus warneri, and
E) Cutibacterium acnes, Corynebacterium afermentans, Corynebacterium tuberculostearicum, Corynebacterium xerosi, Micrococcus luteus, Staphylococcus aureus, Staphylococcus capitis, Staphylococcus epidermidis Staphylococcus hominis, Staphylococcus warneri, and Streptococcus mitis.

4. The method according to claim 1, wherein the synthetic mixed culture of a) comprises a eukaryotic species.

5. The method according to claim 1, wherein cultivation in b) is conducted with the bacterial strains resembling the skin microbiome being at least in part in suspension.

6. The method according to claim 1, wherein cultivation in b) is conducted anaerobically.

7. The method according to claim 1, wherein cultivation in b) is conducted aerobically at low agitation rates of less than 800 rpm.

8. The method according to claim 1, wherein cultivation step b) is conducted in laboratory dishes.

9. The method according to claim 1, wherein the diversity level and/or the diversity profile of the microbiome is measured by 16S rRNA analysis. cm 10. The method according to claim 1, wherein the control microbiome in c) is a synthetic mixed reference culture cultivated identically to said synthetic mixed culture but without the compound and said synthetic mixed culture at the beginning of b).

11. A synthetic mixed culture, comprising at least 8 isolated, different bacterial strains resembling a skin microbiome.

12. The synthetic mixed culture according to claim 11, wherein the bacterial strains are selected from the group consisting of Cutibacterium acnes, Corynebacterium afermentans, Corynebacterium amycolatum, Corynebacterium aurimucosum, Corynebacterium fastidiosum, Corynebacterium kroppenstedtii, Corynebacterium resistens, Corynebacterium simulans, Corynebacterium striatum, Corynebacterium tuberculostearicum, Corynebacterium xerosi, Enhydrobacter aerosaccus, Micrococcus luteus, Propionibacterium acnes, Staphylococcus aureus, Staphylococcus capitis, Staphylococcus epidermidis, Staphylococcus haemolyticus, Staphylococcus hominis, Staphylococcus warneri. Streptococcus mitis, Streptococcus oralis, Streptococcus pseudopneumoniae, Streptococcus sanguinis, and Veillonella parvula.

13. The method according to claim 1, wherein the at least 8 different bacterial strains comprises at least 10 bacterial strains.

14. The method according to claim 1, wherein the at least 8 different bacterial strains comprises at least 12 bacterial strains.

15. The method according to claim 4, wherein the eukaryotic species is from Malassezia, Aspergillus, Debaroyomyces, or Cryptococcus.

16. The method according to claim 4, wherein the eukaryotic species is M. restricta or M. globose.

17. The method according to claim 7, wherein the agitation rates are from 300 rpm to 600 rpm.

18. The method according to claim 8, wherein the laboratory dishes are multi well plates.

19. The synthetic mixed culture according to claim 11, wherein the at least 8 isolated, different bacterial strains comprises at least 10 bacterial strains.

20. The synthetic mixed culture according to claim 11, wherein the at least 8 isolated, different bacterial strains comprises at least 12 bacterial strains.


SEQUENCE LISTING (2 sequences)

SEQ ID NO: 1 - DNA, 17 bp, Artificial Sequence
  Primer
  misc_feature (9)..(9): n is a, c, g, or t
  misc_feature (13)..(13): w is a or t
cctacgggnggcwgcag

SEQ ID NO: 2 - DNA, 21 bp, Artificial Sequence
  Primer
  misc_feature (7)..(7): h is a, c, or t
  misc_feature (8)..(8): v is a, c, or g
  misc_feature (19)..(19): k is g or t
gactachvgggtatctaakcc